(19) United States
(12) Patent Application Publication
(10) Pub. No.: US 2005/0125897 A1
(43) Pub. Date: Jun. 16, 2005

(54) Method for the preparation of apparatuses to be used for the performance of medical or surgical operations

(75) Inventors: Ulrich Wyslucha, Karlsruhe (DE); Michael Welsch, Gaggenau (DE)

Correspondence Address: MCDONNELL BOEHNEN HULBERT & BERGHOFF LLP, 300 S. WACKER DRIVE, 32ND FLOOR, CHICAGO, IL, 60606, US

(73) Assignee: MAQUET GmbH & Co. KG, Rastatt (DE)

(21) Appl. No.: 10/990,021

(22) Filed: Nov. 16, 2004

(30) Foreign Application Priority Data
Nov. 18, 2003 (DE) DE 103 53 846.1

(51) Int. Cl. G09G 5/00; A61B 5/08; A47B 71/00

(52) U.S. Cl. 5/600; 345/156

(57) ABSTRACT

A method for the preparation and/or provision of apparatuses to be used for the performance of medical or surgical operations is characterized in that at a workstation which is connected to a data processing device and comprises at least a display device and a control panel an operation to be performed is called up through the control panel, whereupon the apparatus or the apparatuses suitable for this purpose and/or their settings or, respectively, their positions relative to one another are displayed on the display device.

This application claims priority to German Patent Application No. 103 53 846.1, filed Nov. 18, 2003, which is hereby incorporated by reference herein in its entirety, including the drawings.

The present invention relates to a method for the preparation and/or provision of apparatuses to be used for the performance of medical or surgical operations.

Usually, patient positions for a specific medical or surgical operation are represented as an image showing the final position in conjunction with explanatory text. Intermediate steps are usually not displayed. In addition, descriptions collected in files are available, which are more or less systematically structured and in which the accessories required for the respective operation as well as their handling are described. The use of this information for the preparation of a specific operation requires a lot of experience and familiarity with the respective apparatus in order to be able to prepare best for the operation.

The invention is based on the object to specify a method of the type mentioned at the beginning, which facilitates the preparation and provision of the apparatuses desired or required for an operation and, at the same time, increases the safety for optimum preparation.

According to the invention, this object is solved in that at a workstation that is connected to a computer and comprises at least one display device and a control panel an operation to be performed is called up through the control panel, whereupon the apparatus or apparatuses suitable or required for this purpose and/or their settings or, respectively, their positions relative to another are displayed on the display device. When, for example, one of the apparatuses is an operating table, whose patient support surface has a plurality of spatially adjustable support surface sections, then the position of the respective support surface sections required for an operation can be displayed. Further, the accessory devices and instruments required for the operation can be displayed, this being done either in image form (still image or video) and/or text form (text or sound). At the same time, external documents can also be integrated.

At the same time, there can be the possibility of displaying the position of the accessory devices at the operating table.

It is particularly helpful when the reaching of a certain setting and/or position of an apparatus or apparatus part is displayed step by step, in that, for example, the steps are reproduced in a specific chronological order or are advanced individually or called up by an operator by making an input at the control panel. In this way, the operator can remember the individual steps as well as the sequence of the measures required for reaching a specific final setting. For this purpose, it is helpful when the settings or positions are not only displayed pictorially but all steps up to the reaching of a final position or setting are simultaneously displayed on the display device in the form of individual images.

When the reaching of a specific setting or final position is not obvious from the design of the apparatus itself, then it can be useful to display the measures required for reaching a certain setting or position as well. In cases of complicated settings, it can be particularly beneficial when the apparatuses and/or their settings and/or positions are displayed in chronological images, and a loose sequence running at larger intervals or a film can be chosen.

In a preferred embodiment of the invention, the workstation is connected to an apparatus to be set, for example an operating table, this operating table being automatically controlled in accordance with the settings and positions displayed. Further, the inventive solution can likewise be used to determine and store the apparatuses and/or the settings, or, respectively positions actually used for the operation. The data stored can be allocated to a patient so that in this way a documentation of the apparatuses and instruments used for the operation as well as of their handling becomes possible. These data can, for example, be stored in the electronic patient file. The input of the data can either take place via a direct connection between the workstation and the respective apparatus, in that the settings of the apparatus are, for example, acquired by sensors. The presence of the required or desired accessory devices as well as their position and setting can take place, for example, via transponders, RFID (Radio Frequency Identifier), switches or magnetic sensors. Likewise, there is the possibility of picking up the apparatuses used as well as their settings or, respectively, positions using a still image camera or a video camera and to forward the required data to the computer, either via a direct link or via a portable storage medium.

Further, the invention relates to a computer software comprising commands and data in encoded form, which, after loading the computer software, cause a computer system to execute the above-described method steps. The computer software usually comprises a user interface and a database. The database can comprise a memory for first data and instructions which are protected by a first access key, as well as a second memory for receiving second data and instructions. This makes it possible to save non-variable data which are, for example, specified by the manufacturer of an operating table system in the first memory, while the second memory offers the user the possibility of specifying own proposals for settings as well as the accessories required for an operation and thus of adapting the computer software to his/her own ideas.

The inventive computer software thus offers the possibility of displaying the individual steps for setting an operating table and the accessory parts required for this purpose on the display device, i.e. usually a display screen. Thus, the positioning progress becomes visible and comprehensible. Images, videos and texts can be used for the representation. It can be useful to manage the texts in an arbitrary number of languages. For each positioning of the patient, an item list of the required accessories can be output. If needed, the list can also be supplied to the electronic patient file. In addition, the individual steps of the patient positioning can be individually supplemented by notes and remarks of the operating personnel. As a result thereof, the user knowledge can be documented and made available. If needed, the user can also define certain favorite settings or positions when the access to elements which are required often is to be accelerated.

The fundamental advantage of the inventive solution is that complex connections are to be displayed in a broken down form and that information can be accessed quickly and safely. At the same time, there is the possibility of linking the program with a program for the stock-keeping of the accessories required for an operation so that the personnel timely receives an information on the available material and its condition, for example, the sterilization condition.

The inventive software can readily be used at a plurality of workstations, which are, for example, connected to a database server as clients. As a result, a central data management is possible which guarantees that the same level of data and knowledge is available at all workstations. This results in less errors and reduced costs.

Thus, the invention further relates to a computer system comprising at least a computer, a display device and a control panel as well as a computer software of the above-described type. The control panel can, for example, be a touch screen, which is particularly suitable for use in an operating room. The system can directly be linked to an apparatus to be set, for example, the operating table, so that when calling up a specific operation the required settings and the accessories needed are not only displayed but the operating table itself can be controlled directly via the computer in order to make the settings provided in the program. On the other hand, each of the settings made can be stored for documentation purposes.

Further features and advantages of the invention result from the dependent claims and the following description which in connection with the enclosed figures explains the invention on the basis of embodiments.

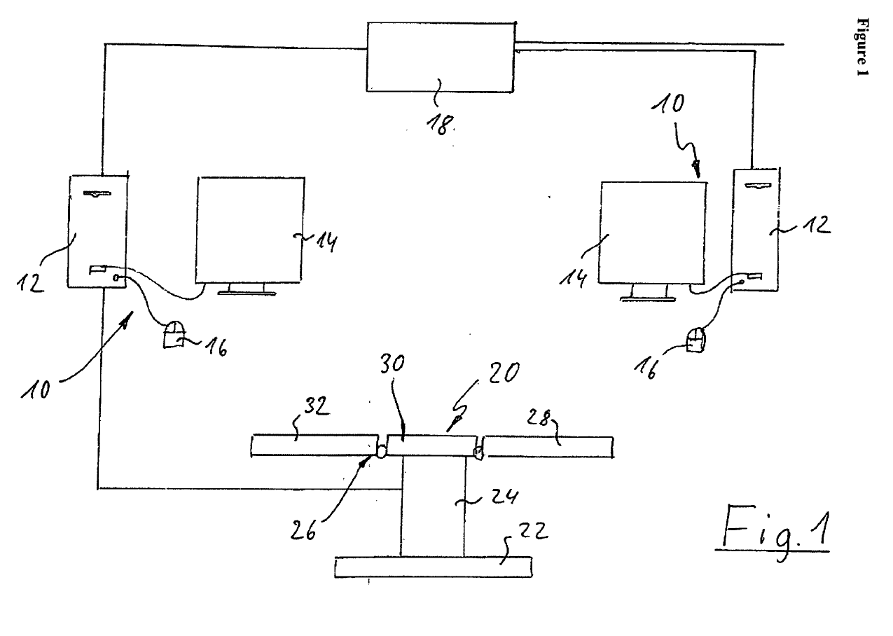

FIG. 1 is a schematic illustration of a computer system suitable for the implementation of the inventive method in connection with a schematically illustrated operating table.

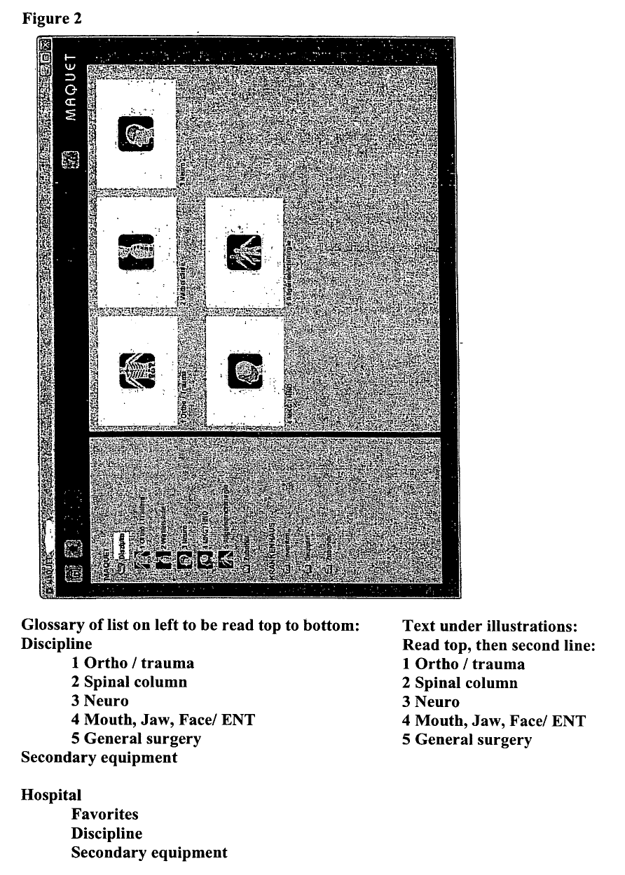

FIGS. 2 to 9 show various images on a display screen for explaining some functions of the inventive computer program.

In FIG. 1, the reference numeral 10 designates each of the workstations comprising a personal computer 12 and a display screen 14 connected thereto. The display screen 14 can be fashioned as a touch screen. Alternatively, the personal computer 12 can be connected to a mouse 16 or a keyboard (not illustrated). The personal computer 12 is connected to a database server 18. Further, in the illustrated embodiment, the personal computer 12 is directly connected to an operating table 20 in order to control the latter. The connection can be a cable connection or a wireless connection. The operating table 20 has a base 22 with a support column 24 and a patient support surface 26 that comprises a plurality of sections 28, 30, 32 which are adjustable relative to one another and to the support column 24 by means of suitable motors. Operating tables of this kind are known and do not have to be explained in detail any more.

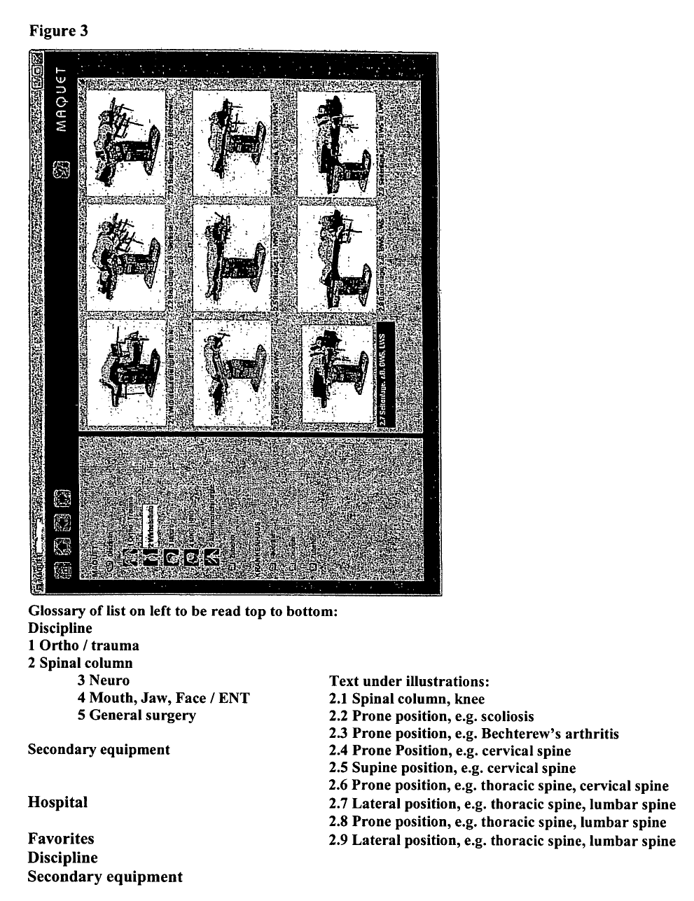

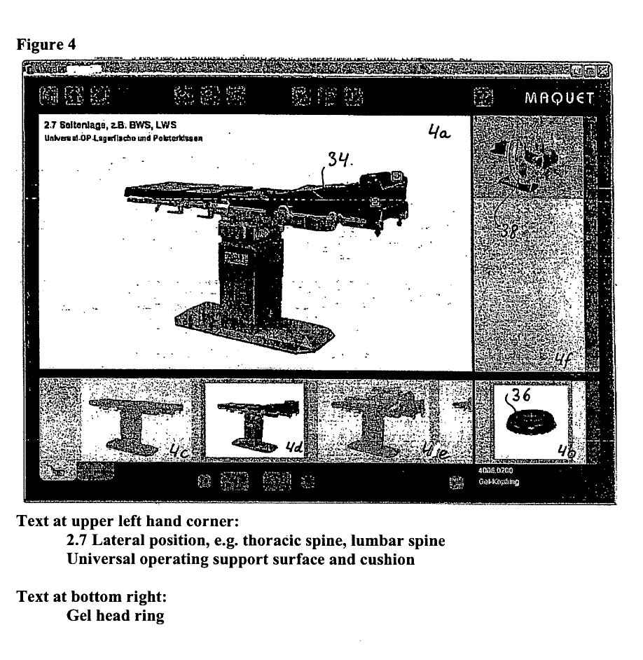

The inventive computer program makes it possible to illustrate to the operating personnel the apparatuses used for a certain operation as well as their settings. Examples thereof are given in FIGS. 2 to 8. FIG. 2 illustrates a first image on the display screen showing a selection of possible disciplines. When one discipline, for example, the spinal column surgery, is chosen, then, according to FIG. 3, various positioning possibilities for different operations are illustrated so that the user can choose the suitable positioning possibility. In FIG. 3, this is the lateral position according to image 2.7. When this lateral position is selected, then in FIG. 4, the operating table is illustrated to the user in the large image 4A with a wedge cushion 34 for the lateral position and a gel head ring 36, which is again illustrated as an accessory part in the small image 4B. The small images 4C to 4E below the large image 4A show the operating table first without any accessories (4C), with the accessories as in 4A (4D) and with the patient in the lateral position (4E). Finally, the image 4F shows the patient in connection with a C-arc 38. Via a link, related patient positions and special applications of a specific patient position can also be displayed here. A fast access to information on accessory parts and additional devices is possible via icons.

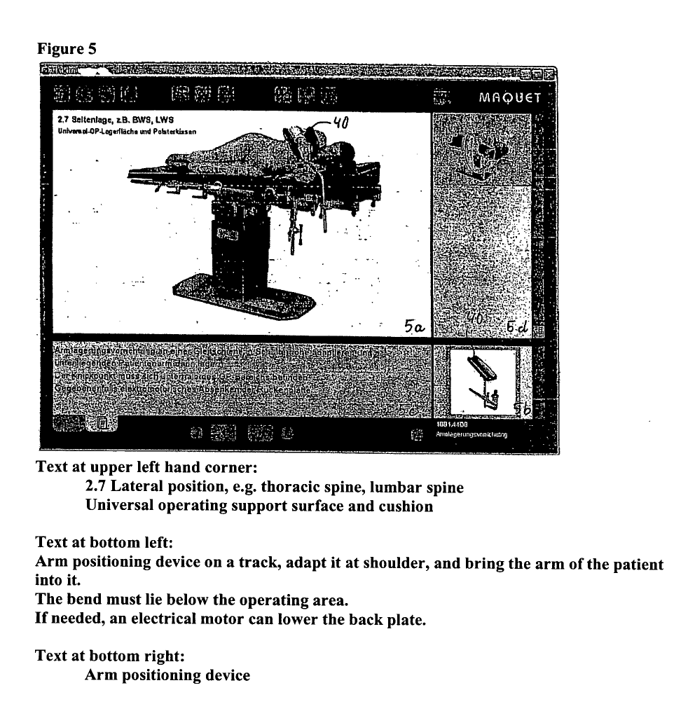

In section 5A, FIG. 5 shows the operating table according to FIG. 4 with the patient and a arm positioning device 40 which is individually illustrated in the image section 5B. The image section 5C provides explanations for the assembly of the arm positioning device. The image section 5D corresponds to the image section 4F.

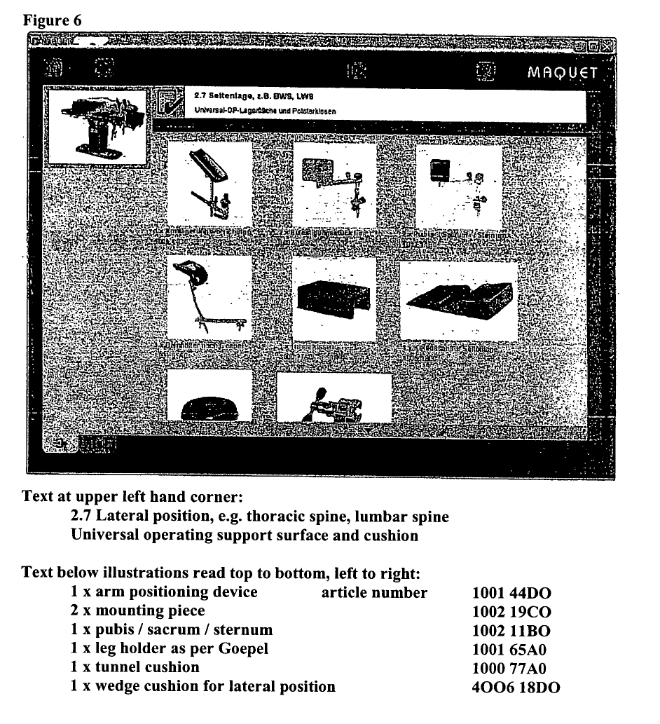

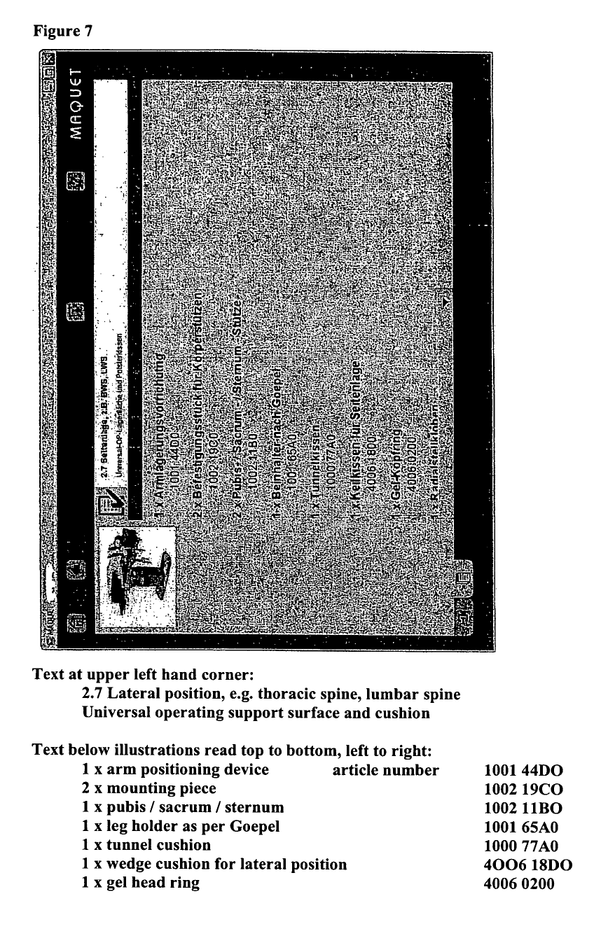

FIG. 6 shows all the accessories required for the lateral position together with item number and quantity. FIG. 7 shows the same accessories in the form of a list, which, for example, can also be printed out.

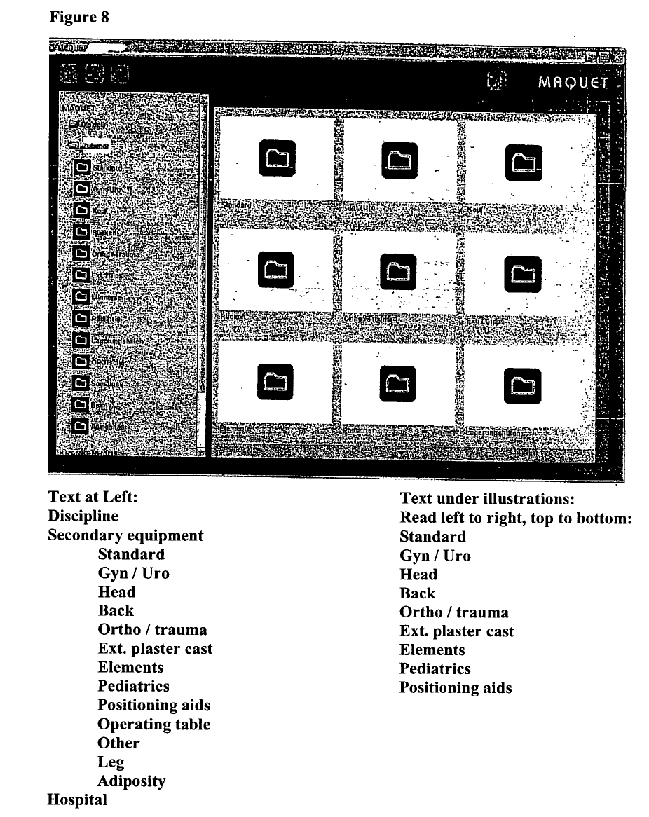

FIG. 8 shows a representation in which the user can select various accessory parts.

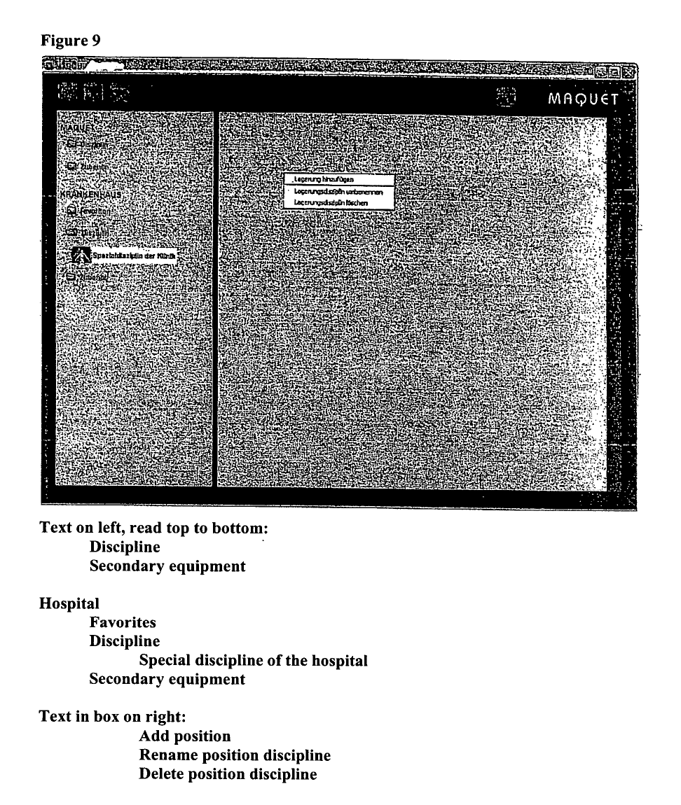

Finally, FIG. 9 shows an image according to which the user can supplement or vary the program with own data in order to adapt it to his/her specific needs or to add own experiences and suggestions.

When an operation is thus prepared on the basis of the instructions of the computer program, then the operating table can be directly controlled and set in accordance with this program, for example, signals being given when a specific accessory part is to be built on and set whereupon, after a corresponding acknowledgement by the user, the program proceeds and provides the next setting. A positioning protocol on this progressive setting can also be recorded. Possibly, accessory parts can also be registered automatically via appropriate transponders or sensors. Positioning protocols that have been created can be printed out or transferred to other programs in order to be able to allocate the respective protocol to the respective patient in a patient management system.

The inventive program and system have a number of advantages. They offer the possibility of interactive training and further education of the hospital personnel. The positioning of a patient can be implemented step by step, the processes being optimized and it being guaranteed that no step is left out. This also results in a higher quality assurance. At the same time, the processes in the operating room can be better documented. The positioning proposals provided by the manufacturer for a certain system can be implemented best. At the same time, each hospital can store own positions and accessories in the program in order to provide a certain standard within the own hospital.

The inventive program can run on conventional data processing units, for example, a personal computer, and can be used by a number of users at the same time. It can be operated via a keyboard or a mouse or a touch screen.

The inventive program enables the networking of information, for example, a listing of all patient positions which make use of specific accessories. Specific manuals of the manufacturer of a system can be fed in, which manuals are easily expandable according to the respective state of the art so that it is guaranteed that the most current version of the manual is always used.

With the inventive system it is easily possible to guarantee that the personnel can completely determine and provide the parts needed for a certain positioning of the patient. At the same time, the parts are illustrated and referenced with the correct name which facilitates the communication among the personnel.

The inventive system has been explained in conjunction with an operating table. However, it is not limited to this application. It can likewise be applied to other devices such as operating lamps and the like.

1. A method for the preparation and/or provision of apparatuses to be used for the performance of medical or surgical operations, comprising a workstation connected to a data processing device and comprising at least a display device and a control panel an operation to be performed is called up through the control panel, whereupon the apparatus or apparatuses and/or their settings and/or their respective positions relative to one another are displayed on the display device.

2. The method according to claim 1, characterized in that one of the apparatuses is an operating table, the patient support surface of which comprises a plurality of spatially adjustable support surface sections, and in that the positions of the support surface sections required for an operation are displayed.

3. The method according to claim 2, characterized in that the accessory devices and instruments required for the operation are displayed.

4. The method according to claim 3, characterized in that the position of the accessory devices at the operating table is displayed.

5. The method according to claim 1, characterized in that the reaching of a certain setting or position of an apparatus or an apparatus part is displayed step by step.

6. The method according to claim 5, characterized in that the steps are advanced by making an input at the control panel.

7. The method according to claims 1, characterized in that the settings or positions are displayed pictorially.

8. The method according to claim 5, characterized in that all steps up to the reaching of a final position or setting are simultaneously displayed in the form of individual images.

9. The method according to claim 1, characterized in that the measures required for reaching a certain setting or position are displayed.

10. The method according to claim 1, characterized in that the apparatuses and/or their settings and/or positions are displayed in chronological images.

11. The method according to claim 1, characterized in that when the workstation is connected to an apparatus to be set, in particular an operating table, this apparatus is automatically controlled in accordance with the settings and positions displayed.

12. The method according to claim 1, characterized in that the apparatuses and settings or, respectively, positions actually used for an operation are determined and stored.

13. The method according to claim 12, characterized in that the stored data is allocated to a patient.

14. A computer software, comprising commands and data in encoded form, which, after loading the computer software, cause a computer system to execute the steps specified in claim 1.

15. The computer software according to claim 14, characterized in that it is stored on a storage medium.

16. The computer software according to claim 14, characterized in that it comprises a user interface and/or a database.

17. The computer software according to claim 16, characterized in that the database comprises a memory with first data and instructions which are protected by a first access key, and in that the database has at least a second memory for receiving further data and instructions.

18. A computer system having at least a computer, a display device and a control panel as well as a computer software according to claim 14.

19. The computer system according to claim 18, characterized in that the control panel is formed on a touch screen forming the display device.

20. The computer system according to claim 18, characterized in that it is connected to an apparatus to be set.

21. The computer system according to claim 18, characterized in that the setting or, respectively, the position of an apparatus to be set or of accessory parts connected thereto can be acquired by sensors and stored in the computer system.

22. The computer system according to claim 20, characterized in that the sensors are transponders connected to the parts to be set or to be positioned.

23. The computer system according to claim 18, characterized in that the sensors comprise at least a video and/or a still image camera.
